SEQUENCE LISTING (39 sequences)

SEQ ID NO: 1 - protein, 355 aa, synthetic construct
  REGION 1..355: note = Description of Artificial Sequence: Synthetic polypeptide
MLTERFNHILLLLIWFRPVSITEVECFLLPLMRKAYLRLDSEISMYSVKVNLEKKAYLRKCKEFTDLGQNLLYTYFSLNNKFMPNRPNYIAFGLRYRSMLLLPGSYTYEWIPYLDGTFYVLAWTRAFVFLGRQLPKLVTEHDTLLYYSEHPTFTSQYNLVPMVATVFPTKDVALQYDPVAALFAYAQKIFKILRPHERNGFTVLELRRKMMYMIPSINVHHYTRATKMQVITTVYPPSSTAKGPISHGHVLKHERNGFTVLCLGGLLTMVGLCTLVAMLSSCSSCPLSKITYGPVFMCLRPPIFIRRLFLRGRAYGLRAKFKQLLHPVGEADYFEYYPLHEQHGMVEITPYKPTW

SEQ ID NO: 2 - protein, 10 aa, polyomavirus BKV
MLTERFNHIL

SEQ ID NO: 3 - protein, 9 aa, polyomavirus BKV
LLLIWFRPV

SEQ ID NO: 4 - protein, 9 aa, polyomavirus BKV
SITEVECFL

SEQ ID NO: 5 - protein, 9 aa, polyomavirus BKV
LPLMRKAYL

SEQ ID NO: 6 - protein, 9 aa, polyomavirus BKV
RLDSEISMY

SEQ ID NO: 7 - protein, 9 aa, polyomavirus BKV
SVKVNLEKK

SEQ ID NO: 8 - protein, 9 aa, polyomavirus BKV
AYLRKCKEF

SEQ ID NO: 9 - protein, 9 aa, unidentified
  REGION 1..9: note = Description of Unknown:Adenovirus sequence
TDLGQNLLY

SEQ ID NO: 10 - protein, 9 aa, unidentified
  REGION 1..9: note = Description of Unknown:Adenovirus sequence
TYFSLNNKF

SEQ ID NO: 11 - protein, 10 aa, unidentified
  REGION 1..10: note = Description of Unknown:Adenovirus sequence
MPNRPNYIAF

SEQ ID NO: 12 - protein, 9 aa, unidentified
  REGION 1..9: note = Description of Unknown:Adenovirus sequence
GLRYRSMLL

SEQ ID NO: 13 - protein, 9 aa, unidentified
  REGION 1..9: note = Description of Unknown:Adenovirus sequence
LPGSYTYEW

SEQ ID NO: 14 - protein, 9 aa, unidentified
  REGION 1..9: note = Description of Unknown:Adenovirus sequence
IPYLDGTFY

SEQ ID NO: 15 - protein, 9 aa, unidentified
  REGION 1..9: note = Description of Unknown:Adenovirus sequence
VLAWTRAFV

SEQ ID NO: 16 - protein, 9 aa, unidentified
  REGION 1..9: note = Description of Unknown:Adenovirus sequence
FLGRQLPKL

SEQ ID NO: 17 - protein, 9 aa, Cytomegalovirus
VTEHDTLLY

SEQ ID NO: 18 - protein, 11 aa, Cytomegalovirus
YSEHPTFTSQY

SEQ ID NO: 19 - protein, 9 aa, Cytomegalovirus
NLVPMVATV

SEQ ID NO: 20 - protein, 8 aa, Cytomegalovirus
FPTKDVAL

SEQ ID NO: 21 - protein, 9 aa, Cytomegalovirus
QYDPVAALF

SEQ ID NO: 22 - protein, 10 aa, Cytomegalovirus
AYAQKIFKIL

SEQ ID NO: 23 - protein, 11 aa, Cytomegalovirus
RPHERNGFTVL

SEQ ID NO: 24 - protein, 9 aa, Cytomegalovirus
ELRRKMMYM

SEQ ID NO: 25 - protein, 9 aa, Cytomegalovirus
IPSINVHHY

SEQ ID NO: 26 - protein, 9 aa, Cytomegalovirus
TRATKMQVI

SEQ ID NO: 27 - protein, 11 aa, Cytomegalovirus
TTVYPPSSTAK

SEQ ID NO: 28 - protein, 10 aa, Cytomegalovirus
GPISHGHVLK

SEQ ID NO: 29 - protein, 9 aa, Cytomegalovirus
HERNGFTVL

SEQ ID NO: 30 - protein, 9 aa, Epstein-Barr virus
CLGGLLTMV

SEQ ID NO: 31 - protein, 9 aa, Epstein-Barr virus
GLCTLVAML

SEQ ID NO: 32 - protein, 11 aa, Epstein-Barr virus
SSCSSCPLSKI

SEQ ID NO: 33 - protein, 9 aa, Epstein-Barr virus
TYGPVFMCL

SEQ ID NO: 34 - protein, 9 aa, Epstein-Barr virus
RPPIFIRRL

SEQ ID NO: 35 - protein, 9 aa, Epstein-Barr virus
FLRGRAYGL

SEQ ID NO: 36 - protein, 8 aa, Epstein-Barr virus
RAKFKQLL

SEQ ID NO: 37 - protein, 11 aa, Epstein-Barr virus
HPVGEADYFEY

SEQ ID NO: 38 - protein, 9 aa, Epstein-Barr virus
YPLHEQHGM

SEQ ID NO: 39 - protein, 10 aa, Epstein-Barr virus
VEITPYKPTW

(19) United States
(12) Patent Application Publication
(10) Pub. No.: US 2023/0068154 A1
(43) Pub. Date: Mar. 2, 2023

(54) MULTIVIRUS-SPECIFIC T CELL IMMUNOTHERAPY

(71) Applicant: THE COUNCIL OF THE QUEENSLAND INSTITUTE OF MEDICAL RESEARCH, Herston (AU)

(72) Inventors: Rajiv KHANNA, Herston (AU); Vijayendra DASARI, The Gap (AU)

(73) Assignee: THE COUNCIL OF THE QUEENSLAND INSTITUTE OF MEDICAL RESEARCH, Herston (AU)

(21) Appl. No.: 17/886,270

(22) Filed: Aug. 11, 2022

Related U.S. Application Data
(63) Continuation of application No. 16/318,555, filed on Jan. 17, 2019, now abandoned.
(60) Provisional application No. 62/363,669, filed on Jul. 18, 2016.

(51) Int. Cl. A61K 39/12; C12N 15/861

(52) U.S. Cl. CPC A61K 39/12; C12N 15/861; C12N 2710/10334; C12N 2710/10343; C12N 2710/16134; C12N 2710/16234; C12N 2710/22034; C12N 2800/22

(57) ABSTRACT

Provided herein are compositions and methods related to a multivirus-specific T cell immunotherapy.

### RELATED APPLICATIONS

This application claims the benefit of priority to U.S. Provisional Patent Application Ser. No. 62/363,669, filed Jul. 18, 2016, hereby incorporated by reference in its entirety.

### SEQUENCE LISTING

The instant application contains a Sequence Listing which has been filed electronically in XML format and is hereby incorporated by reference in its entirety. Said XML copy, created on Nov. 3, 2022, is named 17-886270_seq_list.xml and is 38,649 bytes in size.

### BACKGROUND

Adoptive immunotherapy involves implanting or infusing disease-specific cytotoxic T cells (CTLs) into individuals with the aim of recognizing, targeting, and destroying disease-associated cells. Adoptive immunotherapy has become a promising route for the treatment of many diseases and disorders, including cancer, infectious diseases and autoimmune diseases.

### SUMMARY

In certain aspects, provided herein are compositions and methods related to the generation and use of multivirus-specific cytotoxic T cells (CTLs) for adoptive immunotherapy. In certain embodiments, provided herein are compositions and methods related to nucleic acids, vectors and recombinant adenoviruses that contain nucleic acid sequences encoding two or more T cell epitopes from different viruses (e.g., as polyepitope proteins) that are recognized by CTLs and that are useful in the prevention and/or treatment of viral infections and/or cancer. In certain embodiments, provided herein are antigen-presenting cells (APCs) that present two or more T cell epitopes from different viruses. In some embodiments, provided herein are populations of CTLs that collectively comprise T cell receptors (TCRs) that recognize two or more T cell epitopes from different viruses.

In certain aspects, provided herein are nucleic acid vectors (e.g., an adenoviral expression vector) and/or recombinant adenoviruses that comprise nucleic acid sequences that encode two or more T cell epitopes (e.g., two or more of the T cell epitopes listed in Table 1), wherein the two or more T cell epitopes comprise T cell epitopes from at least two different viruses (e.g., Epstein Barr virus (EBV), cytomegalovirus (CMV), polyoma BK virus (BKV) and/or adenovirus (ADV)). In some embodiments, the epitopes are HLA class I-restricted T cell epitopes. In some embodiments, the vector or recombinant adenovirus encodes for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 T cell epitopes (e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 of the epitopes listed in Table 1).

In some embodiments, the vector or recombinant adenovirus encodes a T cell epitope from EBV (e.g., an LMP2a epitope, an EBNA3A epitope, an EBNA3B epitope, an EBNA1 epitope, a BZLF1 epitope, and/or BMLF1 epitope). In some embodiments, the vector or recombinant adenovirus encodes a T cell epitope from CMV (e.g., a pp50 epitope, a pp65 epitope, an IE-1 epitope, and/or a pp150 epitope). In some embodiments, the vector or recombinant adenovirus encodes a T cell epitope from BKV (e.g., a large T antigen epitope and/or a VP1 epitope). In some embodiments, the vector or recombinant adenovirus encodes a T cell epitope from ADV (e.g., a hexon protein epitope, a DNA polymerase epitope, and/or DNA binding protein epitope). In some embodiments, the T cell epitopes comprise epitopes from at least three or four different viruses (e.g., Epstein Barr Virus (EBV), cytomegalovirus (CMV), polyoma BK virus (BKV), and adenovirus (ADV)). In some embodiments, the vector or recombinant adenovirus may encode T cell epitopes from any combination of the aforementioned viruses and/or from other viruses. In some embodiments, the vector or recombinant adenovirus encodes for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 T cell epitopes (e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 of the epitopes listed in Table 1). In some embodiments, the T cell epitopes encoded by the vectors or recombinant adenovirus described herein are encoded as a polyepitope protein (i.e., a single chain of amino acid residues comprising multiple T cell epitopes not directly linked in nature). In some aspects, the polyepitope protein comprises an amino acid sequence that has at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 1. In some embodiments, the sequences encoding the T cell epitopes (e.g., the T cell epitopes in the polyepitope protein) are codon optimized.

In some aspects, provided herein are methods of generating a recombinant adenoviruses disclosed herein. In some embodiments, the method includes transfecting a nucleic acid vector described herein into a cell line (e.g., HEK 293 cells) and then culturing the transfected cell line under conditions such that the cell line produces the recombinant adenovirus. In some embodiments, the method further includes isolating the recombinant adenovirus.

In some aspects, provided herein are therapeutic compositions (vaccine compositions or other pharmaceutical compositions), comprising the vectors, recombinant adenoviruses, or polyepitopes disclosed herein, and methods of treating or preventing viral infections or cancer using the therapeutic compositions.

In some aspects, provided herein are APCs that present two or more T cell epitopes (e.g., two or more of the T cell epitopes listed in Table 1), wherein the two or more T cell epitopes comprise T cell epitopes from at least two different viruses (e.g., Epstein Barr virus (EBV), cytomegalovirus (CMV), polyoma BK virus (BKV) and/or adenovirus (ADV)). In some embodiments, the epitopes are HLA class I-restricted T cell epitopes. In some embodiments, the APCs present a T cell epitope from EBV (e.g., an LMP2a epitope, an EBNA3A epitope, an EBNA3B epitope, an EBNA1 epitope, a BZLF1 epitope, and/or BMLF1 epitope). In some embodiments, the APCs present a T cell epitope from CMV (e.g., a pp50 epitope, a pp65 epitope, an IE-1 epitope, and/or a pp150 epitope). In some embodiments, the APCs present a T cell epitope from BKV (e.g., a large T antigen epitope and/or a VP1 epitope). In some embodiments, the APCs present a T cell epitope from ADV (e.g., a hexon protein epitope, a DNA polymerase epitope, and/or DNA binding protein epitope). In some embodiments, the T cell epitopes comprise epitopes from at least three or four different viruses (e.g., Epstein Barr Virus (EBV), cytomegalovirus (CMV), polyoma BK virus (BKV), and adenovirus (ADV)). In some embodiments, the APCs present T cell epitopes from any combination of the aforementioned viruses and/or from other viruses. In some embodiments, APCs present at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 T cell epitopes (e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 of the epitopes listed in Table 1).

In some aspects, provided herein is a population of CTLs collectively comprise T cell receptors that recognize two or more T cell epitopes (e.g., two or more of the T cell epitopes listed in Table 1), wherein the two or more T cell epitopes comprise T cell epitopes from at least two different viruses (e.g., Epstein Barr virus (EBV), cytomegalovirus (CMV), polyoma BK virus (BKV) and/or adenovirus (ADV)). In some embodiments, the epitopes are HLA class I-restricted T cell epitopes. In some embodiments, the population of CTLs collectively comprise T cell receptors that recognize a T cell epitope from EBV (e.g., an LMP2a epitope, an EBNA3A epitope, an EBNA3B epitope, an EBNA1 epitope, a BZLF1 epitope, and/or BMLF1 epitope). In some embodiments, the population of CTLs collectively comprise T cell receptors that recognize a T cell epitope from CMV (e.g., a pp50 epitope, a pp65 epitope, an IE-1 epitope, and/or a pp150 epitope). In some embodiments, the population of CTLs collectively comprise T cell receptors that recognize a T cell epitope from BKV (e.g., a large T antigen epitope and/or a VP1 epitope). In some embodiments, the population of CTLs collectively comprise T cell receptors that recognize a T cell epitope from ADV (e.g., a hexon protein epitope, a DNA polymerase epitope, and/or DNA binding protein epitope). In some embodiments, the population of CTLs collectively comprise T cell receptors that recognize T cell epitopes from at least three or four different viruses (e.g., Epstein Barr Virus (EBV), cytomegalovirus (CMV), polyoma BK virus (BKV), and adenovirus (ADV)). In some embodiments, the population of CTLs collectively comprise T cell receptors that recognize T cell epitopes from any combination of the aforementioned viruses and/or from other viruses. In some embodiments, population of CTLs collectively comprise T cell receptors that recognize at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 T cell epitopes (e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 of the epitopes listed in Table 1).

In some aspects, provided herein are methods of generating antigen APCs that present multi-virus T cell epitopes. In some embodiments, the method includes transfecting APCs with a vector provided herein. In some embodiments, the method includes contacting the APCs with a recombinant adenovirus provided herein. In some embodiments, the APCs are B cells, antigen-presenting T-cells, dendritic cells, and/or artificial antigen-presenting cells (e.g., aK562 cells). In some aspects, provided herein are methods of generating, activating and/or inducing proliferation of multivirus-specific CTLs that recognize two or more of the T cell epitopes described herein, for example, by incubating a sample comprising CTLs (e.g., a PBMC sample) with APCs described herein. In some embodiments, provided herein are APCs and/or T cells generated according to the methods described herein.

In some aspects, provided herein are methods of treating and/or preventing viral infection (e.g., EBV, CMV, BKV, or ADV) and/or cancer by administering to a subject a composition comprising the CTLs described herein. In some embodiments, the subject is immunocompromised. In some embodiments, the CTLs are autologous to the subject. In some embodiments, the CTLs are allogeneic to the subject. In some embodiments, the CTLs are stored in a cell bank prior to administration to the subject. In some embodiments, CTLs are selected (e.g., selected from a cell bank) for compatibility with the subject prior to administration to the subject. In some embodiments, the CTLs are selected if they are restricted through an HLA allele shared with the subject (i.e., the TCR of the CLTs are restricted to an MHC class I protein encoded by a HLA allele that is present in the subject). In some embodiments, the CTLs are selected if the CTLs and subject share at least 2 (e.g., at least 3, at least 4, at least 5, at least 6) HLA alleles and the CTLs are restricted through a shared HLA allele. In some embodiments, the CTLs administered to the subject are selected from a cell bank (e.g., a CTL bank).

### BRIEF DESCRIPTION OF THE DRAWINGS

FIG. 1 shows a schematic depicting an exemplary method for the construction of an exemplary adenoviral nucleic acid vector followed by the use of such a vector for the generation of an exemplary recombinant adenovirus (Ad-MvP). According to this exemplary method, synthetic DNA sequence encoding a polyepitope protein containing contiguous HLA class I-restricted CTL epitopes from BKV, ADV, CMV and EBV was cloned into a pShuttle vector and then subcloned into the Ad5F35 expression vector. The recombinant Ad5F35 vector was packaged into infectious adenovirus by transfecting HEK 293 cells, and recombinant adenovirus (referred to as Ad-MvP) was harvested from transfected cells by repeated freeze-thawing cycles.

FIG. 2 shows expansion of multivirus-specific T cells from solid-organ transplant recipients with the exemplary nucleic acid vector. PBMC from 14 SOT patients were stimulated with Ad-MvP and cultured for 14 days in the presence of IL-2. The frequency of epitope specific CTL was determined by measuring IFNγ production in response to stimulation with virus-specific peptide pools containing epitopes encoded in Ad-MvP. A: Representative dot plots following recall with CMV, EBV, BKV or ADV peptide epitopes is shown. B: Data represents a summary of the number of virus-specific IFNγ-producing CD8⁺ T cells from all SOT patients. Black symbols represent patients recruited with CMV-associated complications, red symbols represent patients with EBV-associated PTLD, and blue symbols represent patients with BKV viremia C: Ad-MvP expanded CTL were assessed for the intracellular production of IFNγ, TNF, IL-2 and externalization of CD107a following in vitro stimulation with the virus-specific peptide pools. Boolean Analysis was performed using FlowJo Software. Pie Charts represent the proportion of T cells specific for each virus capable of generating 1, 2, 3 or 4 effector functions.

FIG. 3 shows priming of multi-virus-specific T cells following immunization. A: Representative data showing ex vivo and in vitro expanded virus-specific T cells from HHD II transgenic mouse immunized with Ad-MvP. B: Stacked bar graph showing percentage of multivirus-specific CD8⁺ T cells expressing IFNγ in HLA*A02 transgenic mice immunized with Ad-MvP. Splenocytes from immunized mice were isolated on day 50 post-vaccination and stimulated in vitro with HLA-A*02-restricted CD8⁺ T cell peptide epitopes from BKV, ADV, CMV or EBV. T cell specificity was assessed using an intracellular cytokine assay.

FIG. 4 shows expansion of multi-virus specific T cells using an exemplary recombinant adenovirus in healthy volunteers. PBMC from healthy volunteers were stimulated with Ad-MvP and expanded in the presence of IL-2 for 14 days. The frequency of epitope specific CTL was determined by measuring IFNγ production in response to stimulation with HLA-matched epitopes contained in Ad-MvP. A: Summary of the frequency of multi-virus specific T cells in a cohort of healthy donors. B: Ad-MvP expanded CTL were stimulated with peptide pools corresponding to the epitopes contained in the polyepitope for each virus. Production of IFNγ, TNF, IL-2 and externalization of CD107a were measured as markers of polyfunctionality. C: In vitro expansion of multivirus-specific CD8⁺ T cells from healthy donors using Ad-MvP in the presence of different cytokine combinations. D: The frequency of antigen-specific T cells following in vitro culture in the presence of different cytokines was assessed using intracellular cytokine assays.

FIG. 5 shows adoptive immunotherapy for EBV-associated B cell lymphoma using an autologous or allogeneic multivirus-specific T cells. A & D: Epitope-specificity analysis of Ad-MvP expanded T cells from donors D01 (HLA A1, A11, B8, B35) and D055 (HLA A1, A2, B8, B40) using intracellular cytokine assays B: NOD/SCID mice (n=10) were engrafted with EBV transformed LCLs from donor H002 to induce B cell lymphoma. On day 6 after engraftment, mice were either mock treated (n=5) or adoptively infused with autologous 2×10⁷ Ad-MvP expanded CTL (n=5; shown in panel A). Tumor volume was measured using vernier calipers. C: Kaplan-Meier survival graph of EBV tumor bearing mice after mock treatment or autologous T cell therapy. E: NOD/SCID mice (n=10) were engrafted with EBV transformed LCL from donor H002 to induce B cell lymphoma. On day 6 after engraftment, mice were either mock treated (n=5) or adoptively infused with HLA matched allogeneic Ad-MvP expanded T cells from donor H005 (n=5; shown in panel B). Tumor volume was measured using vernier calipers. Each data points in panels B & E shows mean±SEM of tumor size as measured in multiple mice using vernier calipers. F: Kaplan-Meier survival graph of EBV tumor bearing mice after mock treatment or allogeneic T cell therapy.

### DETAILED DESCRIPTION

### General

In certain aspects, provided herein are compositions and methods related to the generation and use of multivirus-specific cytotoxic T cells (CTLs) for adoptive immunotherapy. In certain embodiments, provided herein are compositions and methods related to nucleic acids, vectors and recombinant adenoviruses that contain nucleic acid sequences encoding two or more T cell epitopes from different viruses (e.g., as polyepitope proteins) that are recognized by CTLs and that are useful in the prevention and/or treatment of viral infections and/or cancer. In certain embodiments, provided herein are antigen-presenting cells (APCs) that present two or more T cell epitopes from different viruses. In some embodiments, provided herein are populations of CTLs that collectively comprise T cell receptors (TCRs) that recognize two or more T cell epitopes from different viruses.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering. Such an agent can contain, for example, peptide described herein, an antigen presenting cell provided herein and/or a CTL provided herein.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

The term “binding” or “interacting” refers to an association, which may be a stable association, between two molecules, e.g., between a TCR and a peptide/MHC, due to, for example, electrostatic, hydrophobic, ionic and/or hydrogen-bond interactions under physiological conditions. A TCR “recognizes” a T cell epitope that it is capable of binding to when the epitope is presented on an appropriate MHC.

The term “biological sample,” “tissue sample,” or simply “sample” each refers to a collection of cells obtained from a tissue of a subject. The source of the tissue sample may be solid tissue, as from a fresh, frozen and/or preserved organ, tissue sample, biopsy, or aspirate; blood or any blood constituents, serum, blood; bodily fluids such as cerebral spinal fluid, amniotic fluid, peritoneal fluid or interstitial fluid, urine, saliva, stool, tears; or cells from any time in gestation or development of the subject.

The term “epitope” means a protein determinant capable of specific binding to an antibody or TCR. Epitopes usually consist of chemically active surface groupings of molecules such as amino acids or sugar side chains. Certain epitopes can be defined by a particular sequence of amino acids to which an antibody is capable of binding.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, a therapeutic that “prevents” a condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

As used herein, the term “subject” means a human or non-human animal selected for treatment or therapy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

The term “vector” refers to the means by which a nucleic acid can be propagated and/or transferred between organisms, cells, or cellular components. Vectors include plasmids, viruses, bacteriophage, pro-viruses, phagemids, transposons, and artificial chromosomes, and the like, that may or may not be able to replicate autonomously or integrate into a chromosome of a host cell.

### Recombinant Adenoviruses and Vectors

In certain aspects, provided herein are nucleic acid molecules (e.g., vectors, such as adenoviral expression vectors) and/or recombinant adenoviruses that comprise nucleic acid sequences that encode two or more T cell epitopes (e.g., two or more of the T cell epitopes listed in Table 1), wherein the two or more T cell epitopes comprise T cell epitopes from at least two different viruses (e.g., Epstein Barr virus (EBV), cytomegalovirus (CMV), polyoma BK virus (BKV) and/or adenovirus (ADV)). In some embodiments, the T cell epitopes are HLA class I-restricted T cell epitopes. For example, the nucleic acid molecules and/or recombinant adenoviruses may comprise nucleic acid sequences encoding T cell epitopes from EBV and CMV, from EBV and BKV, from EBV and ADV, from CMV and ADV, from CMV and BKV, or from BKV and ADV. In some embodiments, the nucleic acid molecules and/or recombinant adenoviruses contain nucleic acid sequences encoding for T cell epitopes from three or more different viruses. For example, the nucleic acid molecules and/or recombinant adenoviruses may comprise nucleic acid sequences encoding T cell epitopes from EBV, CMV and BKV, from EBV, CMV and ADV, from CMV, BKV and ADV, or from ADV, BKV and EBV. n some embodiments, the nucleic acid molecules and/or recombinant adenoviruses contain nucleic acid sequences encoding for T cell epitopes from three or more different viruses. For example, the nucleic acid molecules and/or recombinant adenoviruses may comprise nucleic acid sequences encoding T cell epitopes from EBV, CMV, BKV, and ADV. In some embodiments, the nucleic acid molecules and/or recombinant adenoviruses may comprise nucleic acid sequences encoding T cell epitopes from 5, 6, 7, 8, 9, 10 or more different viruses. In some embodiments, the sequences encoding the T cell epitopes (e.g., the T cell epitopes in the polyepitope protein) are codon optimized.

In some embodiments, the T cell epitopes encoded by the vectors or recombinant adenovirus described herein are encoded as a polyepitope protein (i.e., a single chain of amino acid residues comprising multiple T cell epitopes not linked in nature). In some embodiments, the T cell epitopes in the polyepitope protein are connected via an amino acid linker. In some embodiments, the T cell epitopes in the polyepitope protein are directly linked without intervening amino acids. An exemplary polyepitope protein amino acid sequence is provided below as SEQ ID NO: 1:

MLTERFNHILLLLIWFRPVSITEVECFLLPLMRKAYLRLDSEISMYSVK
VNLEKKAYLRKCKEFTDLGQNLLYTYFSLNNKFMPNRPNYIAFGLRYRS
MLLLPGSYTYEWIPYLDGTFYVLAWTRAFVFLGRQLPKLVTEHDTLLYY
SEHPTFTSQYNLVPMVATVFPTKDVALQYDPVAALFAYAQKIFKILRPH
ERNGFTVLELRRKMMYMIPSINVHHYTRATKMQVITTVYPPSSTAKGPI
SHGHVLKHERNGFTVLCLGGLLTMVGLCTLVAMLSSCSSCPLSKITYGP
VFMCLRPPIFIRRLFLRGRAYGLRAKFKQLLHPVGEADYFEYYPLHEQH
GMVEITPYKPTW

In some aspects, the polyepitope protein comprises an amino acid sequence that has at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 1. To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

In some embodiments, the nucleic acid molecules and/or recombinant adenoviruses provided herein comprise a nucleic acid sequence encoding 2 or more, 3 or more, 4 or more, 5 or more, 6 or more, 7 or more, 8 or more, 9 or more, 10 or more, 11 or more, 12 or more, 13 or more, 14 or more, 15 more, 16 or more, 17 or more, 18 or more, 19 or more, 20 or more, 21 or more, 22 or more, 23 more, 24 or more, 25 or more, 26 or more, 27 or more, 28 or more, 29 or more, 30 or more, 31 or more, 32 or more, 33 or more, 34 or more, 35 or more, 36 or more, 38 or more, 39 or more, or 40 or more T cell epitopes. In some embodiments, the T cell epitopes comprise a T cell epitope from EBV (e.g., an LMP2a epitope, an EBNA3A epitope, an EBNA3B epitope, an EBNA1 epitope, a BZLF1 epitope, and/or a BMLF1 epitope). In some embodiments, the T cell epitopes comprise a T cell epitope from CMV (e.g., a pp50 epitope, a pp65 epitope, an IE-1 epitope, and/or a pp150 epitope). In some embodiments, T cell epitopes comprise a T cell epitope from BKV (e.g. a large T antigen epitope and/or a VP1 epitope) In some embodiments, the T cell epitopes comprise a T cell epitope from ADV (e.g., a hexon protein epitope, a DNA polymerase epitope, and/or DNA binding protein epitope).

In some embodiments, the nucleic acid molecules and/or recombinant adenoviruses provided herein comprise a nucleic acid sequence encoding a T cell epitope provided in Table 1. In some embodiments, the nucleic acid vector or recombinant adenoviral expression vector comprises all of the epitopes listed in Table 1. In some embodiments, the nucleic acid molecules and/or recombinant adenoviruses provided herein comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 of the T cell epitopes listed in Table 1.

TABLE 1
List of Exemplary HLA class I restricted T cell epitopes.

| Virus | Sequence^{#} | Antigen | HLA Restriction | SEQ ID NO |
|---|---|---|---|---|
| BKV | MLTERFNHIL | large T antigen | A*02 | 2 |
| | LLLIWFRPV | large T antigen | A*02:01 | 3 |
| | SITEVECFL | VP1 | A*02:01 | 4 |
| | LPLMRKAYL | large T antigen | B*07:02, B*08 | 5 |
| | RLDSEISMY | large T antigen | A*01 | 6 |
| | SVKVNLEKK | large T antigen | A*03 | 7 |
| | AYLRKCKEF | large T antigen | A*24 | 8 |
| ADV | TDLGQNLLY | hexon protein | A*01 | 9 |
| | TYFSLNNKF | hexon protein | A*24:02 | 10 |
| | MPNRPNYIAF | hexon protein | B*07, B*35 | 11 |
| | GLRYRSMLL | hexon protein | A*02:02 | 12 |
| | LPGSYTYEW | hexon protein | B*53:01 | 13 |
| | IPYLDGTFY | hexon protein | B*35, B*53:01 | 14 |
| | VLAWTRAFV | DNA polymerase | A*02 | 15 |
| | FLGRQLPKL | DNA Binding Protein | A*02 | 16 |
| CMV | VTEHDTLLY | pp50 | A*01 | 17 |
| | YSEHPTFTSQY⁺ | pp65 | A*01, B*44 | 18 |
| | NLVPMVATV | pp65 | A*02:01 | 19 |
| | FPTKDVAL | pp65 | B*35:02, B*35:08 | 20 |
| | QYDPVAALF | pp65 | A*24:02 | 21 |
| | AYAQKIFKIL | IE-1 | A*23:01, A*24:02 | 22 |
| | RPHERNGFTVL | pp65 | B*07:02 | 23 |
| | ELRRKMMYM | IE-1 | B*08:01 | 24 |
| | IPSINVHHY | pp65 | B*35:01 | 25 |
| | TRATKMQVI | pp65 | C*06:02 | 26 |
| | TTVYPPSSTAK | pp150 | A*03:01, A*68:01 | 27 |
| | GPISHGHVLK | pp65 | A*11 | 28 |
| | HERNGFTVL | pp65 | B*40:01 | 29 |
| EBV | CLGGLLTMV | LMP2a | A*02:01 | 30 |
| | GLCTLVAML | BMLF1 | A*02:01 | 31 |
| | SSCSSCPLSKI | LMP2a | A*11:01 | 32 |
| | TYGPVFMCL | LMP2a | A*24:02 | 33 |
| | RPPIFIRRL | EBNA3A | B*07:02 | 34 |
| | FLRGRAYGL | EBNA3A | B*08:01 | 35 |
| | RAKFKQLL | BZLF1 | B*08:01 | 36 |
| | HPVGEADYFEY⁺ | EBNA1 | B*35:01, B*35:08, | 37 |
| | | | B*53:01 | |
| | YPLHEQHGM | EBNA3A | B*35:01, B*35:02, | 38 |
| | | | B*35:03 | |
| | VEITPYKPTW | EBNA3B | B*44:02 | 39 |

In some aspects, provided herein are vectors (e.g., an adenovirus based expression vector) that contain the nucleic acid molecules described herein. As used herein, the term “vector,” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication, episomal mammalian vectors). Other vectors (e.g., non-episomal mammalian vectors) can be integrated into the genome of a host cell upon introduction into the host cell, and thereby be replicated along with the host genome. Moreover, certain vectors are capable of directing the expression of genes. Such vectors are referred to herein as “recombinant expression vectors” (or simply, “expression vectors”). In some embodiments, provided herein are nucleic acids operable linked to one or more regulatory sequences (e.g., a promoter) in an expression vector. In some embodiments the cell transcribes the nucleic acid provided herein and thereby expresses an antibody, antigen binding fragment thereof or peptide described herein. The nucleic acid molecule can be integrated into the genome of the cell or it can be extrachromosomal.

In some embodiments, the nucleic acid vectors or recombinant adenoviruses provided herein consist of two or more epitopes from at least two different viruses listed in Table 1. In some embodiments, the nucleic acid vectors or recombinant adenoviruses provided herein encoded for essentially an epitope listed in Table 1. In some embodiments, the nucleic acid vectors or recombinant adenoviruses provided herein encoded for no more than 20, 19, 18, 17, 16, 15, 14, 13, 12, 11, 10, 9, 8, 7, 6, 5, 4, 3, 2 or 1 amino acids in addition to the epitopes listed in Table 1.

In some embodiments, the sequence of the T cell epitopes comprise an epitope sequence provided herein except for 1 or more (e.g., 1, 2, 3, 4 or 5) conservative sequence modifications. As used herein, the term “conservative sequence modifications” is intended to refer to amino acid modifications that do not significantly affect or alter the interaction between a TCR and a peptide containing the amino acid sequence presented on an MHC. Such conservative modifications include amino acid substitutions, additions (e.g., additions of amino acids to the N or C terminus of the peptide) and deletions (e.g., deletions of amino acids from the N or C terminus of the peptide). Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues of the peptides described herein can be replaced with other amino acid residues from the same side chain family and the altered peptide can be tested for retention of TCR binding using methods known in the art. Modifications can be introduced into an antibody by standard techniques known in the art, such as site-directed mutagenesis and PCR-mediated mutagenesis.

Also provided herein are chimeric or fusion proteins (e.g., polyepitope proteins). As used herein, a “chimeric protein” or “fusion protein” comprises a peptide(s) provided herein (e.g., peptides comprising an epitope listed in Table 1) linked to a distinct peptide to which it is not linked in nature. For example, the distinct peptide can be fused to the N-terminus or C-terminus of the peptide either directly, through a peptide bond, or indirectly through a chemical linker. In some embodiments, the peptide of the provided herein is linked to polypeptides comprising other T cell epitopes. In some embodiments, the peptide provided herein is linked to peptides comprising epitopes from other viral and/or infectious diseases. In some embodiments, the polyepitope provided herein is linked to a peptide encoding a cancer-associated epitope.

A chimeric or fusion peptide provided herein can be produced by standard recombinant DNA techniques. For example, DNA fragments coding for the different peptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, for example, Current Protocols in Molecular Biology, Ausubel et al., eds., John Wiley & Sons: 1992). Moreover, many expression vectors are commercially available that already encode a fusion moiety.

In some embodiments, the nucleic acid vectors or recombinant adenoviruses comprise nucleic acid sequences that have undergone codon optimization. In such embodiments a coding sequence is constructed by varying the codons in each nucleic acid used to assemble the coding sequence. In general, a method to identify a nucleotide sequence that optimizes codon usages for production of a peptide comprises at least the following steps (a) through (e). In step (a), oligomers are provided encoding portions of the polypeptide containing degenerate forms of the codon for an amino acid encoded in the portions, with the oligomers extended to provide flanking coding sequences with overlapping sequences. In step (b), the oligomers are treated to effect assembly of the coding sequence for the peptide. The reassembled peptide is included in an expression system that is operably linked to control sequences to effect its expression. In step (c), the expression system is transfected into a culture of compatible host cells. In step (d), the colonies obtained from the transformed host cells are tested for levels of production of the polypeptide. In step (e), at least one colony with the highest or a satisfactory production of the polypeptide is obtained from the expression system. The sequence of the portion of the expression system that encodes the protein is determined. Further description of codon optimization is provided in U.S. Patent Publication number US2010/035768, which is incorporated by reference in its entirety.

In some embodiments, the nucleic acid vectors, recombinant adenoviruses, or polyepitopes provided herein are part of a vaccine. In some embodiments, the vaccine is delivered to a subject in a vector, including, but not limited to, a bacterial vector and/or a viral vector. Examples of bacterial vectors include, but are not limited to, Mycobacterium bovis (BCG), Salmonella Typhimurium ssp., Salmonella Typhi ssp., Clostridium sp. spores, Escherichia coli Nissle 1917, Escherichia coli K-12/LLO, Listeria monocytogenes, and Shigella flexneri. Examples of viral vectors include, but are not limited to, vaccinia, adenovirus, RNA viruses (replicons), and replication-defective like avipox, fowlpox, canarypox, MVA, and adenovirus.

In some embodiments, provided herein are cells that contain nucleic acid vectors or recombinant adenoviruses described herein. The cell can be, for example, prokaryotic, eukaryotic, mammalian, avian, murine and/or human. In some embodiments, the cell is a mammalian cell. In some embodiments, the cell may be HEK 293 cells. In some embodiments, the cell is an APC (e.g., an antigen-presenting T cell, a dendritic cell, a B cell, or an aK562 cell). In the present methods, nucleic acid vectors or recombinant adenoviruses described herein can be administered to the cell, for example, as nucleic acid without delivery vehicle, in combination with a delivery reagent. In some embodiments, any nucleic acid delivery method known in the art can be used in the methods described herein. Suitable delivery reagents include, but are not limited to, e.g., the Mirus Transit TKO lipophilic reagent; lipofectin; lipofectamine; cellfectin; polycations (e.g., polylysine), atelocollagen, nanoplexes and liposomes. In some embodiments of the methods described herein, liposomes are used to deliver a nucleic acid to a cell or subject. Liposomes suitable for use in the methods described herein can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

### Cells

In some aspects, provided herein are APCs that present on MHC two or more T cell epitopes (e.g., two or more of the T cell epitopes listed in Table 1), wherein the two or more T cell epitopes comprise T cell epitopes from at least two different viruses (e.g., Epstein Barr virus (EBV), cytomegalovirus (CMV), polyoma BK virus (BKV) and/or adenovirus (ADV)). In some embodiments, the MHC is a class I MHC. In some embodiments, the MHC is a class II MHC. In some embodiments, the class I MHC has an a chain polypeptide that is HLA-A, HLA-B, HLA-C, HLA-E, HLA-F, HLA-g, HLA-K or HLA-L. In some embodiment, the class II MHC has an a chain polypeptide that is HLA-DMA, HLA-DOA, HLA-DPA, HLA-DQA or HLA-DRA. In some embodiments, the class II MHC has a R chain polypeptide that is HLA-DMB, HLA-DOB, HLA-DPB, HLA-DQB or HLA-DRB. In some embodiments, APCs present at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 T cell epitopes (e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 of the epitopes listed in Table 1).

In some embodiments the APCs are B cells, antigen presenting T-cells, dendritic cells, or artificial antigen-presenting cells (e.g., aK562 cells). Dendritic cells for use in the process may be prepared by taking PBMCs from a patient sample and adhering them to plastic. Generally the monocyte population sticks and all other cells can be washed off. The adherent population is then differentiated with IL-4 and GM-CSF to produce monocyte derived dendritic cells. These cells may be matured by the addition of IL-1β, IL-6, PGE-1 and TNF-α (which upregulates the important co-stimulatory molecules on the surface of the dendritic cell) and are then contacted with a recombinant adenovirus described herein.

In some embodiments, the APC is an artificial antigen-presenting cell, such as an aK562 cell. In some embodiments, the artificial antigen-presenting cells are engineered to express CD80, CD83, 41BB-L, and/or CD86. Exemplary artificial antigen-presenting cells, including aK562 cells, are described U.S. Pat. Pub. No. 2003/0147869, which is hereby incorporated by reference.

In certain aspects, provided herein are methods of generating APCs that present the two or more of the T cell epitopes described herein comprising contacting an APC with a nucleic acid vector and/or recombinant adenoviruses encoding T cell epitopes described herein and/or with a polyepitope produced by the nucleic acid vectors or recombinant adenoviruses described herein. In some embodiments, the APCs are irradiated.

In some aspects, provided herein are methods of generating, activating and/or inducing proliferation of T cells (e.g., CTLs) that recognize two or more T cell epitopes from at least two different viruses. In some embodiments, the CTLs are incubated in culture with an APC provided herein (e.g., an APC that presents a peptide comprising a T cell epitope). In some embodiments, the sample containing T cells are incubated 2 or more times with APCs provided herein. In some embodiments, the T cells are incubated with the APCs in the presence of at least one cytokine. In some embodiments, the cytokine is IL-4, IL-7 and/or IL-15. Exemplary methods for inducing proliferation of T cells using APCs are provided, for example, in U.S. Pat. Pub. No. 2015/0017723, which is hereby incorporated by reference.

In some aspects, provided herein is a population of CTLs collectively comprising T cell receptors that recognize two or more T cell epitopes (e.g., two or more of the T cell epitopes listed in Table 1), wherein the two or more T cell epitopes comprise T cell epitopes from at least two different viruses (e.g., Epstein Barr virus (EBV), cytomegalovirus (CMV), polyoma BK virus (BKV) and/or adenovirus (ADV)). In some embodiments, the epitopes are HLA class I-restricted T cell epitopes. In some embodiments, the population of CTLs collectively comprise T cell receptors that recognize a T cell epitope from EBV (e.g., an LMP2a epitope, an EBNA3A epitope, an EBNA3B epitope, an EBNA1 epitope, a BZLF1 epitope, and/or BMLF1 epitope). In some embodiments, the population of CTLs collectively comprise T cell receptors that recognize a T cell epitope from CMV (e.g., a pp50 epitope, a pp65 epitope, an IE-1 epitope, and/or a pp150 epitope). In some embodiments, the population of CTLs collectively comprise T cell receptors that recognize a T cell epitope from BKV (e.g., a large T antigen epitope and/or a VP1 epitope). In some embodiments, the population of CTLs collectively comprise T cell receptors that recognize a T cell epitope from ADV (e.g., a hexon protein epitope, a DNA polymerase epitope, and/or DNA binding protein epitope). In some embodiments, the population of CTLs collectively comprise T cell receptors that recognize T cell epitopes from at least three or four different viruses (e.g., Epstein Barr Virus (EBV), cytomegalovirus (CMV), polyoma BK virus (BKV), and adenovirus (ADV)). In some embodiments, the population of CTLs collectively comprise T cell receptors that recognize T cell epitopes from any combination of the aforementioned viruses and/or from other viruses. In some embodiments, the population of CTLs collectively comprise T cell receptors that recognize at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 T cell epitopes (e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 of the epitopes listed in Table 1).

In some aspects, provided herein are compositions (e.g., therapeutic compositions) comprising the nucleic acid vector described herein, peptides produced by the nucleic acid vector described herein, multivirus-specific CTLs and/or APCs provided herein (e.g., comprising the nucleic acid vector described herein) and a pharmaceutically acceptable carrier. In some embodiments, such compositions are used in adoptive immunotherapy to boost multi-virus-specific immunity in a subject by administering to the subject an effective amount of the composition. In some embodiments, the multivirus-specific CTLs and/or APCs are not autologous to the subject. In some embodiments, the T cells and/or APCs are autologous to the subject. In some embodiments, the T cells and/or APCs are stored in a cell bank before they are administered to the subject.

### Pharmaceutical Compositions

In some aspects, provided herein are compositions (e.g., a pharmaceutical composition), containing a nucleic acid vector, a recombinant adenoviruses, a polyepitope protein, a CTL and/or an APC provided herein. In some embodiments, the composition includes a combination of multiple (e.g., two or more) agents provided herein.

In some embodiments, the pharmaceutic compositions provided herein are vaccine compositions. In some embodiments, the pharmaceutical composition further comprises an adjuvant. As used herein, the term “adjuvant” broadly refers to an agent that affects an immunological or physiological response in a patient or subject. For example, an adjuvant might increase the presence of an antigen over time or to an area of interest like a tumor, help absorb an antigen-presenting cell antigen, activate macrophages and lymphocytes and support the production of cytokines. By changing an immune response, an adjuvant might permit a smaller dose of an immune interacting agent to increase the effectiveness or safety of a particular dose of the immune interacting agent. For example, an adjuvant might prevent T cell exhaustion and thus increase the effectiveness or safety of a particular immune interacting agent. Examples of adjuvants include, but are not limited to, an immune modulatory protein, Adjuvant 65, α-GalCer, aluminum phosphate, aluminum hydroxide, calcium phosphate, β-Glucan Peptide, CpG DNA, GPI-0100, lipid A, lipopolysaccharide, Lipovant, Montanide, N-acetyl-muramyl-L-alanyl-D-isoglutamine, Pam3CSK4, quil A and trehalose dimycolate.

### Therapeutic Methods

In certain aspects, provided herein are methods of treating or preventing a viral infection (e.g., a EBV, CMV, BKV, or ADV infection) and/or a cancer in a subject comprising administering to the subject a pharmaceutical composition provided herein.

In some embodiments, provided herein is a method of or preventing treating a viral infection in a subject (e.g., a EBV, CMV, BKV, or ADV infection). In some embodiments, the subject treated is immunocompromised. For example, in some embodiments, the subject has a T cell deficiency. In some embodiments, the subject has leukemia, lymphoma or multiple myeloma. In some embodiments, the subject is infected with HIV and/or has AIDS. In some embodiments, the subject has undergone a tissue, organ and/or bone marrow transplant. In some embodiments, the subject is being administered immunosuppressive drugs. In some embodiments, the subject has undergone and/or is undergoing chemotherapy. In some embodiments, the subject has undergone and/or is undergoing radiation therapy.

In some embodiments, the subject has cancer. In some embodiments, the methods described herein may be used to treat any cancerous or pre-cancerous tumor. In some embodiments, the cancer expresses one or more of the T cell epitopes provided herein (e.g., the T cell epitopes listed in Table 1). In some embodiments, the cancer includes a solid tumor. Cancers that may be treated by methods and compositions provided herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the subject is also administered an immune checkpoint inhibitor. Immune Checkpoint inhibition broadly refers to inhibiting the checkpoints that cancer cells can produce to prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA. Immune checkpoint inhibitors can be antibodies or antigen binding fragments thereof that bind to and inhibit an immune checkpoint protein. Examples of immune checkpoint inhibitors include, but are not limited to, nivolumab, pembrolizumab, pidilizumab, AMP-224, AMP-514, STI-A1110, TSR-042, RG-7446, BMS-936559, MEDI-4736, MSB-0020718C, AUR-012 and STI-A1010.

In some embodiments, a composition provided herein (e.g., a vaccine composition provided herein) is administered prophylactically to prevent cancer and/or a viral infection. In some embodiments, the vaccine is administered to inhibit tumor cell expansion. The vaccine may be administered prior to or after the detection of cancer cells or virally infected cells in a patient. Inhibition of tumor cell expansion is understood to refer to preventing, stopping, slowing the growth, or killing of tumor cells. In some embodiments, after administration of a vaccine comprising nucleic acid vectors, recombinant adenoviruses, polyepitopes, CTLs or APCs described herein, a proinflammatory response is induced. The proinflammatory immune response comprises production of proinflammatory cytokines and/or chemokines, for example, interferon gamma (IFN-7) and/or interleukin 2 (IL-2). Proinflammatory cytokines and chemokines are well known in the art.

### EXAMPLES

### Materials and Methods

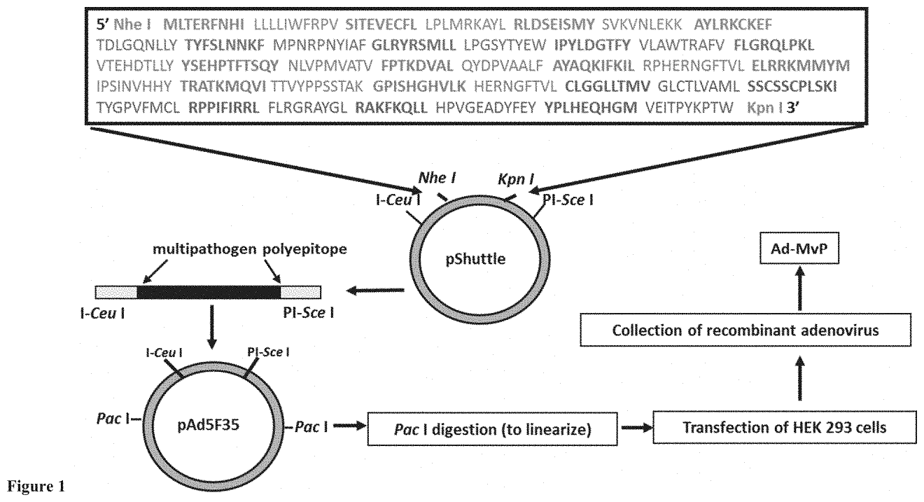

Construction of multivirus adenoviral vector (Ad-MvP). The amino acid sequence of the 32 contiguous HLA class-I restricted CD8⁺ T cell epitopes as a polyepitope from CMV, EBV ADV and BKV (Table 1) was translated into the nucleotide sequence using human universal codon usage. The nucleotide acid sequence encoding the polyepitope with Nhe I and Kpn I restriction sites at 5′ and 3′ respectively was cloned into the pShuttle expression vector. Following amplification, the expression cassette from pShuttle was subcloned into an Ad5F35 expression vector. The recombinant Ad5F35 vector was transfected into human embryonic kidney HEK293 cells, and recombinant adenovirus (referred to as Ad-MvP) stocks were produced in HEK293 cells (FIG. 1).

In vitro expansion of multivirus-specific T-cells. Peripheral blood mononuclear cells (PBMCs) were isolated from peripheral blood by Ficoll gradient, washed and resuspended in RPMI-1640 supplemented with 10% FBS (growth medium) or revived from frozen stocks and rested for at least 1 h at 37° C. before being used in T cell assays. The cells were divided into responder and stimulator cells at a responder to stimulator ratio of 2:1. The stimulator cells were infected with Ad-MvP at a multiplicity of infection of 10:1 for 1 h at 37° C. Unbound virus particles were washed off and the stimulator cells were co-cultured with the responder cells in the presence of different cytokines as indicated (interleukin-2, IL-2-120 IU/ml, IL-21-30 ng/ml, IL-7-10 ng/ml and/or IL-15-10 ng/ml). Every 3 to 4 days, the cultures were supplemented with growth medium containing the respective cytokines. Virus-specific T cell expansion was tested on day 14 using an intracellular cytokine assay.

Characterization of multi-virus specific CTL by intracellular cytokine assay and flow cytometry. PBMCs or cultured T-cells were stimulated with 1 μg/ml peptides corresponding to defined HLA class I-restricted CD8⁺ T-cell epitopes derived from CMV, EBV, BKV or ADV proteins and incubated in the presence of a CD107a-antibody, Brefeldin A and Monensin for 5 h. After surface staining for CD8 and CD4, cells were fixed and permeabilized with cytofix/cytoperm and stained for IFNγ, IL-2 and TNF. Stained cells were resuspended in PBS containing 2% paraformaldehyde and acquired using a FACSCanto II or LSR Fortessa with FACSDiva software (BD Biosciences). Post-acquisition analysis was conducted using FlowJo software (TreeStar).

Ad-MvP immunisation in HLA transgenic mice. All animal immunisation protocols were conducted in compliance with the QIMR Berghofer Medical Research Institute Animal Ethics Committee. HLA-A*02 transgenic mice (HHD II) were maintained in a pathogen-free animal facility at QIMR Berghofer. Three groups (placebo, prime, prime-boost) of six to eight week old female mice were injected intramuscularly with 50 μl PBS or 50 μl Ad-MvP (1×10⁹ pfu/mL). A booster dose was given on day 21 to the prime-boost group. Mice were sacrificed on day 50, splenocytes from all the groups were stimulated in vitro with BKV, ADV, CMV or EBV-specific HLA-A*02 restricted peptide pools. Splenocytes were cultured in a 24 well plate for 10 days at 37° C., 10% CO₂. On days 3 and 6, cultures were supplemented with growth medium containing recombinant IL-2. T cell specificity was assessed using an intracellular cytokine staining assay.

Adoptive transfer of multi-virus specific T cells in an EBV lymphoma model. Two groups of adult (6-10 week-old) NOD/SCID mice irradiated with a single dose of 230 cGy were engrafted subcutaneously with 10⁷ EBV-transformed lymphoblastoid cells (LCLs) per mouse. Tumour growth was monitored every 2-3 days using vernier callipers. Six days after engraftment of LCLs, mice were either mock treated or infused with 2×10⁷ Ad-MvP-expanded T cells. These in vitro-expanded T cells included EBV-, CMV-, ADV- and BKV-specific T cells. Tumour burden was monitored after adoptive T cell therapy and mice were sacrificed when tumour volume reached 1000 m³.

Statistical analysis. The group difference between mice treated with Ad-MvP-expanded autologous or allogeneic antigen-specific T cells and mock-treated mice was evaluated by a linear mixed-effect model with time, group and the interaction of time and group as predictors.

### Example 1: Single Stimulation with an Exemplary Nucleic Acid Vector (Ad-MvP) is Sufficient to Expand Polyfunctional Multi-Virus Specific T Cells from Transplant Recipients

In order to explore the potential application of the Ad-MvP antigen presentation system (FIG. 1) for transplant recipients, a cohort of SOT recipients who had either ongoing or a previous history of recurrent viral reactivation/disease (CMV, EBV or BKV) was recruited. Clinical characteristics of SOT patients can be found in Table 2.

TABLE 2
Clinical characteristics of SOT recipients

| Patient | | | Serological | Antiviral | CMV/EBV/BKV | CMV/EBV/BKV |
|---|---|---|---|---|---|---|
| ID | Organ | Drugs | status | treatment | reactivations post tx | disease |
| SOT02 | lung | FK, MMF, | R+/D+ | Val | 2 (CMV) | Yes (eye) |
| | | P | (CMV) | | | |
| SOT06 | lung | CsA. MMF, | R+/D+ | Gan | 2 (CMV) | Yes (lung) |
| | | P | ICMV) | Val | | |
| SOT26 | lung | FK, MMF, | R+/D+ | Val | 2 (CMV) | No |
| | | P | (CMV) | | | |
| $OT35 | lung | CsA. MMF, | R-/D+ | Val | 1 (CMV) | No |
| | | PFK | (CMV) | | | |
| SOT56 | kidney | TK, MMF, | R+/D- | Val | 0 | No |
| | | P | (CMV) | | | |
| SOTS8 | kidney | FK, MMF. | R-/D+ | Gan | 1 (CMV) | No |
| | | PB | (CMV) | | | |
| SOT62 | kidney | CsA, MMF, | R+/D- | None | 3 (CMV) | No |
| | | P, FK | (CMV) | | | |
| SOT68 | kidney | CsA, MMF, | R-/D+ | Val | 2 (CMV) | Ko |
| | | P | (CMV) | | | |
| SOT75 | kidney | CsA, MMF, | R-/D+ | Gan, Val | 3 (CMV) | No |
| | | P, FK | (CMV) | | | |
| SOT22 | Lung | CsA.P. | R-/D+ | Gan | 2 (EBV) | Yes |
| | | MMF | | | | (PTLD) |
| | | AZA | | | | |
| SOT33 | Heart | CsA, P | R-/D+ | Val | 2 (EBV) | Yes |
| | | AZA | (EBV) | | | (PTLD) |
| SOT59 | Lung | CsA, AZA, | R-/D+ | NA | 1 (EBV) | Yes |
| | | P | (EBV) | | | (PTLD) |
| SOH5 | Kidney | FK, P, E | R-/D+ | None | 1 (BKV) | Yes |
| | | | (BKV) | | | (BKVAN) |
| $OT22 | Kidney | FK, P | R-/D+ | Lef | 1 (BKV) | Yes |
| | | | (BKV) | | | (BKVAN) |

Abbreviations: tx—transplantation, R—recipient, D—donor, Gan—ganciclovir, Val—valganciclovir, FK—tacrolimus, P—prednisone, CsA—cyclosporin A, MMF—mycophenolate mofetil, E—Everolimus , Lef—Leflunomide, AZA—azathioprine B—basiliximab; PTLD—post-transplant lymphoproliferative disorder, BKVAN—BK-associated nephropathy

Peripheral blood mononuclear cells (PMBCs) from these SOT recipients were stimulated with Ad-MvP. A schematic outline for the construction of Ad-MvP can be found in FIG. 1. Synthetic DNA sequence encoding a polyepitope protein containing contiguous 32 HLA class I-restricted CTL epitopes from BKV, ADV, CMV and EBV was cloned into a pShuttle vector and then subcloned into the Ad5F35 expression vector. The recombinant Ad5F35 vector was packaged into infectious adenovirus by transfecting HEK 293 cells, and recombinant adenovirus (referred to as Ad-MvP) was harvested from transfected cells by repeated freeze-thawing cycles.

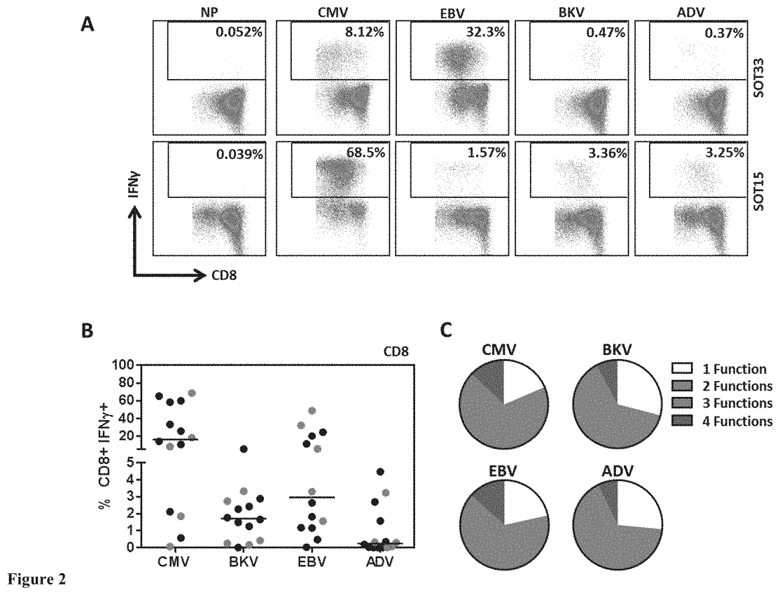

Peripheral blood mononuclear cells (PMBCs) from these SOT recipients were stimulated with Ad-MvP at a multiplicity of infection (MOI) 10:1 and then cultured for 14 days. Representative data from two different transplant recipients presented in FIG. 2A shows that a single stimulation with Ad-MvP was sufficient to induce the rapid expansion of T cells specific for ADV, BKV, CMV and EBV epitopes. T cells expanded from SOT33 showed strong reactivity towards CMV and EBV, while T cells expanded from SOT15 showed strong reactivity against CMV but also EBV, BKV and ADV. A comprehensive summary of T cell expansions following Ad-MvP stimulation from 14 SOT recipients is presented in FIG. 2B. These analyses showed that CMV, BKV, EBV and ADV-specific T cell expansions were observed in 86%, 71%, 86% and 29% of SOT patients respectively (FIG. 2B). More importantly, the majority of these in vitro expanded T cells showed a polyfunctional profile (FIG. 2C). Taken together, these studies showed that Ad-MvP is highly efficient in expanding multivirus-specific T cells from transplant recipients and this expansion is not impacted by underlying immunosuppression or ongoing viral reactivation/disease.

### Example 2: In Vivo Priming of Multivirus-Specific T Cells with Ad-MvP

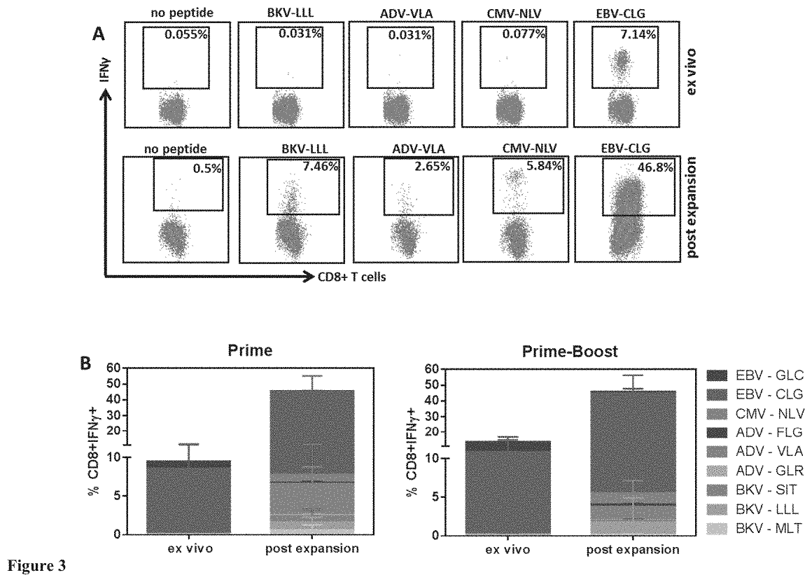

In addition to the potential application of Ad-MvP as a tool for in vitro expansion of pre-existing memory/effector T cells, a humanized mouse model was also used to explored the utility of this vector for in vivo priming of multivirus-specific T cells in seronegative transplant recipients/donors. Humanized transgenic mice expressing the HLA A*0201 allele (referred to as HHD II mice) were immunized with Ad-MvP (0.5×10⁸ pfu/mouse) and then one group was boosted with the same dose on day 21. On day 50 post-immunization, these mice were assessed for antigen-specific T cell responses. While ex vivo analysis revealed strong T cell response to EBV epitopes and low or undetectable response towards epitopes from CMV, BKV and ADV, a 6-240 fold increase in antigen-specific T cells was observed following in vitro stimulation with BKV, ADV, CMV or EBV-specific HLA-A*0201-restricted peptide pools (FIG. 3A). A comprehensive summary of multiple HLA-A2-restricted T cell responses in HHD II mice following Ad-MvP prime alone and prime-boost immunization is shown in FIG. 3B. This analysis also showed that while in both the prime alone and prime-boost setting EBV-specific T cell responses were the dominant component of ex vivo analysis, a significant change in the composition of antigen-specific T cells was observed following in vitro stimulation. Taken together, these experiments clearly demonstrated that Ad-MvP vector is highly efficient in inducing multivirus-specific T cells in vivo.

### Example 3: Expansion of Multivirus-Specific T Cells from Healthy Donors with Ad-MvP for Third-Party T Cell Bank

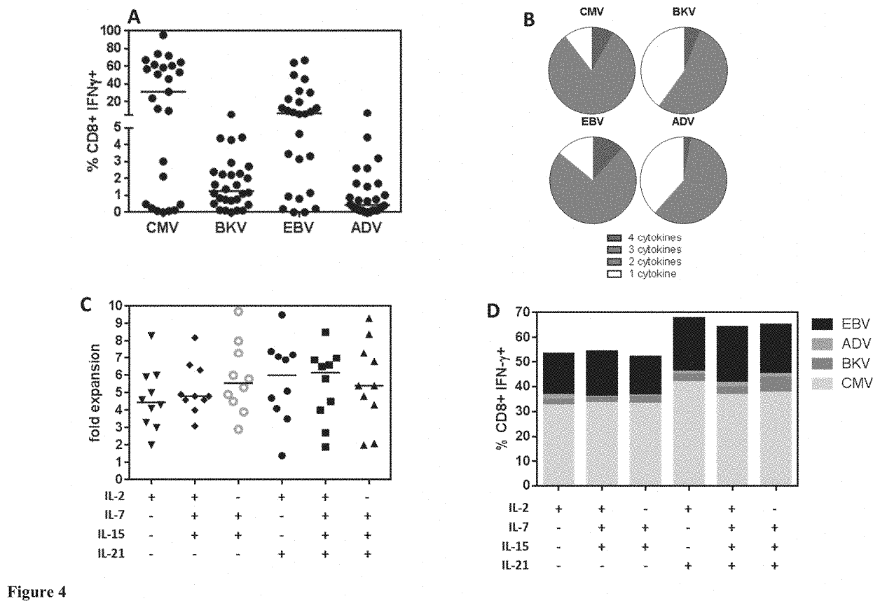

While autologous T cell therapy has been successfully used to treat many SOT recipients, many patients are not amenable to this therapy due to severe lymphopenia or transplant-related clinical complications. More recently, third-party HLA matched virus-specific T cell therapy has emerged as an excellent alternative to autologous cellular therapy. To assess AD-MvP as a potential tool for manufacturing T cell banks, PBMCs from a panel of healthy volunteers were stimulated with autologous PBMCs infected with Ad-MvP at a MOI of 10:1 and then cultured for 14 days. A comprehensive summary of T cell expansions following Ad-MvP stimulation from 20 healthy donors is presented in FIG. 4A. These analyses showed that in all healthy donor samples T cells specific for at least three different viruses were detected. The mean expansions of CD8⁺ IFNγ⁺ T cells specific for CMV, EBV, BKV and ADV were 33.83%, 15.91%, 1.70% and 1.12% respectively. The polyfunctional profiling of these in vitro expanded effector cells showed that 60-80% of EBV, CMV, BKV and ADV-specific T cells showed coincident expression of IFNγ, TNF and/or IL-2 with strong cytotoxic potential as assessed by CD107a mobilization (FIG. 4B).

To further refine the culture conditions required for optimal yield of multivirus-specific T cells, T cell expansion potential was assessed in the presence of different cytokine combinations in comparison to the standard supplementation with IL-2 alone. PBMCs from healthy donors were stimulated with Ad-MvP and expanded in the presence of combinations of IL-2, IL-21, IL-7 and/or IL-15/IL-7. While the overall T cell expansions and polyfunctional profile was slightly improved when cells were cultured in the presence of IL-2 in combination with IL-21 and IL-15, there was no statistically significant difference when compared to T cell expansion in IL-2 alone (FIGS. 4C & D).

### Example 4: Autologous and Allogeneic Adoptive Immunotherapy with Ad-MvP-Expanded T Cells

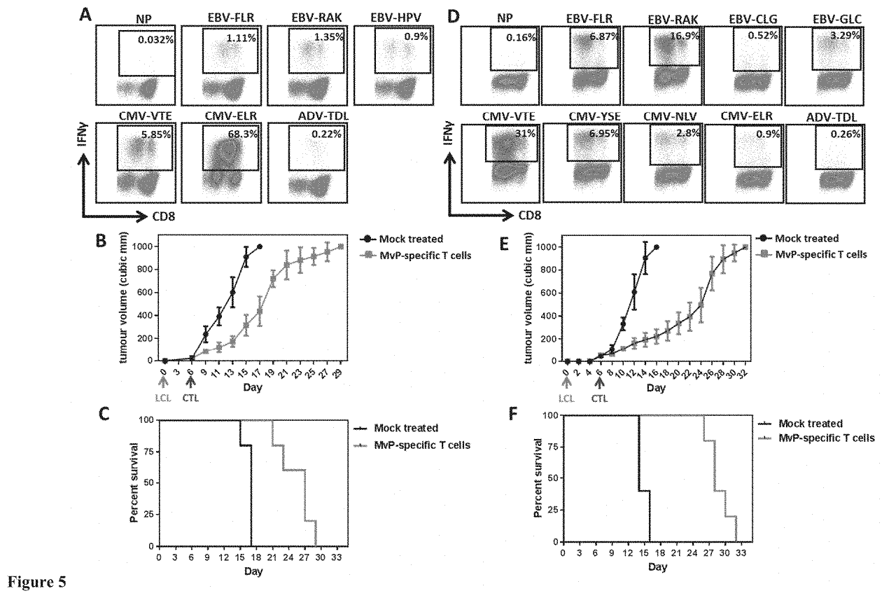

Having established the in vitro and in vivo immunogenicity of the Ad-MvP vector, the next set of experiments were designed to assess the potential therapeutic application of the Ad-MvP vector in a humanized mouse model of EBV-associated lymphoma. A group of immunodeficient NOD/SCID mice were engrafted with EBV-transformed LCLs (Donor code: D01; HLA A1, A11, B8 and B35). Autologous T cells from D01 were expanded using Ad-MvP and which included CD8⁺ T cells specific for three EBV epitopes (HLA B8 and B35-restricted) as well as CMV and ADV (FIG. 5A). On day 6 after EBV lymphoma induction, mice were adoptively treated with a single injection of autologous Ad-MvP expanded T cells. Data presented in FIGS. 5B & C shows that following adoptive immunotherapy, a significant delay in lymphoma outgrowth was observed in mice treated with Ad-MvP-expanded autologous T cells when compared to mock-treated mice (p=0.033). Considering the broader applicability of allogeneic antigen-specific T cell therapy, therapeutic efficacy of Ad-MvP expanded T cells from a HLA-matched donor (Donor code: D055; HLA A1, A2, B8 and B40) was assessed. The expanded T cells from D055 included T cells specific for CMV, ADV and four EBV epitopes restricted through HLA B8 and HLA A2. T cells specific for HLA B8-restricted epitopes (FLR and RAK) matched to the EBV lymphoma in NOD/SCID mice (FIG. 5D; p=0.0065). Tumor bearing mice treated with allogeneic multivirus-specific T cells also showed significantly delayed tumor growth (FIGS. 5E and 5F).

What is claimed is:

1. A cytotoxic T cell (CTL) population, collectively comprising T cell receptors that recognize two or more of the T cell epitopes, wherein the two or more T cell epitopes comprise T cell epitopes from at least two different viruses.

2. The CTL population of, wherein the two or more T cell epitopes are HLA class I restricted T cell epitopes.

3. The CTL population of claim 1, wherein the T cell receptors recognize at least 3, 5, 10, 15, 20, 25, or 30 of the T cell epitopes listed in Table 1.

4. The CTL population of claim 1, wherein the T cell epitopes comprise T cell epitopes from at least three different viruses.

5. The CTL population of claim 1, wherein the T cell epitopes comprise T cell epitopes from at least four different viruses.

6. The CTL population of claim 1, wherein the T cell receptors recognize a T cell epitope from Epstein Barr virus (EBV).

7. The CTL population of claim 6, wherein the T cell epitope from EBV comprises one or more of an EBNA1 epitope, a BZLF1 epitope, a LMP2 epitope, a EBNA3 epitope, or a BMLF1 epitope.

8. The CTL population of claim 1, wherein the T cell receptors recognize a T cell epitope from cytomegalovirus (CMV).

9. The CTL population of claim 8, wherein the T cell epitope from CMV comprises one or more of an IE-1 epitope, a pp65 epitope, a pp150 epitope, or a pp50 epitope.

10. The CTL population of claim 1, wherein the T cell receptors recognize a T cell epitope from polyoma BK virus (BKV).

11. The CTL population of claim 25, wherein the T cell epitope from BKV is one or both of a large T antigen epitope, and a VP1 epitope.

12. The CTL population of claim 1, wherein the T cell receptors recognize a T cell epitope from adenovirus (ADV).

13. The CTL population of claim 12, wherein the T cell epitope from ADV is a hexon protein epitope.

14. The CTL population of claim 1, wherein the T cell receptors recognize T cell epitopes from EBV, CMV and BKV.

15. The CTL population of claim 1, wherein the T cell receptors recognize T cell epitopes from EBV, CMV and ADV.

16. The CTL population of claim 1, wherein the T cell receptors recognize T cell epitopes from CMV, BKV and ADV.

17. The CTL population of claim 11, wherein the T cell receptors recognize T cell epitopes from ADV, BKV and EBV.

18. The CTL population of claim 1, wherein the T cell receptors recognize T cell epitopes from EBV, CMV, BKV and ADV.

19. The CTL population of claim 1, wherein the T cell receptors recognize T cell epitopes from EBV, CMV, BKV and ADV.

20. A cytotoxic T cell (CTL) population, collectively comprising T cell receptors that recognize a plurality of T cell epitopes, wherein the plurality of T cell epitopes comprise T cell epitopes from at least EBV, CMV, BKV and ADV, and wherein the T cell epitopes from EBV comprise an EBNA1 epitope, a BZLF1 epitope and an LMP2 epitope, the T cell epitopes from CMV comprise an IE-1 epitope and a pp65 epitope, the T cell epitopes from BKV comprise large T antigen epitope and a VP1 epitope, and the T cell epitope from ADV comprises a hexon protein epitope.
